(19) United States
(12) Patent Application Publication
(10) Pub. No.: US 2022/0202627 A1
(43) Pub. Date: Jun. 30, 2022

(54) SENSOR UNIT, ABSORBENT ARTICLE KIT, AND ABSORBENT ARTICLE PACKAGE

(71) Applicant: UNICHARM CORPORATION, Ehime (JP)

(72) Inventors: Shota Uto, Kagawa (JP); Tomomi Isogai, Kagawa (JP); Takuya Miyama, Kagawa (JP)

(73) Assignee: UNICHARM CORPORATION, Ehime (JP)

(21) Appl. No.: 17/697,116

(22) Filed: Mar. 17, 2022

Related U.S. Application Data
(63) Continuation of application No. PCT/JP2020/038599, filed on Oct. 13, 2020.

(30) Foreign Application Priority Data
Nov. 1, 2019 (JP) 2019-200040

(51) Int. Cl. A61F 13/42; A61F 13/551; A61F 13/49

(52) U.S. Cl. CPC A61F 13/42; A61F 2013/422; A61F 13/49001; A61F 13/551

(57) ABSTRACT

A sensor unit for an absorbent article includes: a urine change-detection unit that detects a change in color of a urine indicator that visually changes a color on contact with urine; and a feces change-detection unit that detects a change in color of a feces indicator that visually changes a color on contact with feces.

### BACKGROUND

### Technical Field

The present invention relates to a sensor unit, an absorbent article kit, and an absorbent-article package.

### Description of Related Art

As an example of absorbent articles, a disposable diaper is given. Some of such disposable diapers have an indicator function of visualizing the amount of excrement absorbed and informing the user of the time for replacement.

For example, Patent Literature 1 discloses a disposable diaper including a urination indicator whose color changes when coming into contact with urine, between an absorbent body and a back-surface sheet of the disposable diaper. In that disposable diaper, the visibility of the indicator is enhanced by compressing an outer-layer nonwoven fabric in a portion that overlaps the indicator.

Patent Literature 1: Japanese Patent Application Publication No. 2012-100886

According to the disposable diaper of Patent Literature 1, a person replacing the diaper can visually recognize from the outside of the diaper that urination has occurred.

However, in the case where the absorbent article includes not only the urine indicator but also a feces indicator whose color is changed due to contact with feces, there is a risk that it makes a person replacing the absorbent article difficult to remember the color tones of the urine indicator and the feces indicator before and after the reaction, or a risk that, in the case where a wearer is wearing clothes over the absorbent article, it makes the replacing person difficult to reliably visually recognize a change in the color of the urine indicator and the feces indicator.

### SUMMARY

One or more embodiments of the present invention provide an absorbent article that makes it possible to easily determine whether the wearer has excreted urine or the wearer has excreted feces.

An aspect of the present invention is a sensor unit for an absorbent article including: a urine change-detection unit that detects a change in color of a urine indicator, the urine indicator being an indicator that visually changes a color due to (on) contact with urine; and a feces change-detection unit that detects a change in color of a feces indicator, the feces indicator being an indicator that visually changes a color due to contact with feces.

Features of one or more embodiments of the present invention other than the above will become clear by reading the description of the present specification with reference to the accompanying drawings.

According to one or more embodiments of the present invention, it makes it easier to determine whether the wearer has excreted urine or the wearer has excreted feces.

### BRIEF DESCRIPTION OF DRAWINGS

FIG. 1 is a plan view of a tape-type disposable diaper 1 in an unfolded and stretched state when viewed from a skin side.

FIG. 2 is a cross-sectional view taken along a line A-A of FIG. 1.

FIGS. 3A, 3B, and 3C each show a state of parts of a feces indicator 40 and urine indicators 50 in FIG. 1 viewed from a non-skin side of the diaper 1.

FIG. 4A is a top view of a sensor unit 80.

FIG. 4B is a side view of the sensor unit 80.

FIG. 4C is a bottom view of the sensor unit 80.

FIG. 5 is a diagram illustrating an example of how the sensor unit 80 is attached.

FIG. 6 is a schematic explanatory diagram illustrating the inside of the sensor unit 80 when viewed from a bottom face side.

FIG. 7 is an explanatory diagram illustrating the principle of measuring the color of the feces indicator 40 by the sensor unit 80.

FIG. 8 is a perspective view of a package 100.

FIG. 9 is a diagram illustrating a modified example of the sensor unit 80.

FIG. 10 is a diagram illustrating a modified example of the sensor unit 80.

### DETAILED DESCRIPTION OF EMBODIMENTS

At least following matters will become clear with description of this specification and attached drawings.

a sensor unit for an absorbent article including: a urine change-detection unit that detects a change in color of a urine indicator, the urine indicator being an indicator that visually changes a color due to contact with urine; and a feces change-detection unit that detects a change in color of a feces indicator, the feces indicator being an indicator that visually changes a color due to contact with feces.

According to the sensor unit, it becomes easily to determine whether the wearer has excreted urine or the wearer has excreted feces.

In such a sensor unit, the absorbent article may further comprise an output unit (or controller), the output unit being a unit that wirelessly outputs detection results detected by the urine change-detection unit and the feces change-detection unit.

According to the sensor unit, the sensor unit can be easily attached and detached, without effort and troubles as in the case of being wired.

In such a sensor unit, letting a direction along a vertical direction of the absorbent article when the sensor unit is attached to the absorbent article be a first direction, letting a direction along a lateral direction of the absorbent article when the sensor unit is attached to the absorbent article be a second direction, with respect to the first direction (the sensor unit is attached to the absorbent article such that, along a vertical direction of the absorbent article), a position of a center of the urine change-detection unit may be different (or offset) from a position of a center of the feces change-detection unit.

According to the sensor unit, it can realize a sensor unit that easily detect a change in the color of the urine indicator and a change in the color of the feces indicator at different positions in the first direction, compared with the case where the position of the center of the urine change-detection unit and the position of the center of the feces change-detection unit are provided at the same position in the first direction.

In such a sensor unit, letting a direction along a vertical direction of the absorbent article when the sensor unit is attached to the absorbent article be a first direction, letting a direction along a lateral direction of the absorbent article when the sensor unit is attached to the absorbent article be a second direction, with respect to the second direction (the sensor unit is attached to the absorbent article such that, along a lateral direction of the absorbent article), a position of a center of the urine change-detection unit may be different (or offset) from a position of a center of the feces change-detection unit.

According to the sensor unit, it can realize a sensor unit that easily detect a change in the color of the urine indicator and a change in the color of the feces indicator at different positions in the second direction, compared with the case where the position of the center of the urine change-detection unit and the position of the center of the feces change-detection unit are provided at the same position in the second direction (in a lateral direction of the absorbent article).

In such a sensor unit, letting a direction along a vertical direction of the absorbent article when the sensor unit is attached to the absorbent article be a first direction, letting a direction along a lateral direction of the absorbent article when the sensor unit is attached to the absorbent article be a second direction, the urine change-detection unit may be provided on each of two sides (or each side) of the feces change-detection unit in the second direction.

According to the sensor unit, even in the case where the sensor unit is attached to an absorbent article in which the urine indicators are provided on two lateral sides of the feces indicator, it is possible to detect a change in the colors of the indicators.

In such a sensor unit, the sensor unit may further comprise an attaching/detaching mechanism, the attaching/detaching mechanism enabling the sensor unit to be attached to or detached from the absorbent article, and the urine change-detection unit and the feces change-detection unit may each have a portion that do not overlap the attaching/detaching mechanism when viewed from a side from which the sensor unit is attached to the absorbent article.

According to the above-described sensor unit, it is possible to prevent hindering by the attaching/detaching mechanism the detection of a change in the colors of the indicators, caused in the case where the attaching/detaching mechanism is provided over the entire region of the urine change-detection unit or the entire region of the feces change-detection unit.

In such a sensor unit, the sensor unit may be flexible.

According to the sensor unit, the texture while the absorbent article is put on is good.

In such a sensor unit, the sensor unit has an attachment side to be attached to the absorbent article and a non-attachment side opposite the attachment side, and the sensor unit has a mark, on the non-attachment side, that indicates an attachment position where the sensor unit is to be attached to the absorbent article.

According to the sensor unit, by attaching the sensor unit to the absorbent article according to the mark, it makes it easier to attach the sensor unit at the correct position of the absorbent article, making it possible to more accurately recognize whether the wearer has excreted urine or the wearer has excreted feces.

In such a sensor unit, the mark may include a urine-indicator mark and a feces-indicator mark, the urine-indicator mark may be provided at a position corresponding to the urine change-detection unit, and the feces-indicator mark may be provided at a position corresponding to the feces change-detection unit.

According to the sensor unit, the sensor unit can be attached at the positions corresponding to the urine indicator and the feces indicator of the absorbent article, making it easier to detect a change in the colors of the indicators.

In such a sensor unit, the urine-indicator mark and the feces-indicator mark may have graphics, and at least one of shapes, patterns, and colors of the graphics may be different between the urine-indicator mark and the feces-indicator mark.

According to the sensor unit, in the sensor unit, it becomes easier to distinguish the positions of the urine indicator and the feces indicator.

In such a sensor unit, a color of the urine-indicator mark may be identical to a color of the urine indicator before the urine indicator is changed due to contact with the urine, and a color of the feces-indicator mark may be identical to a color of the feces indicator before the feces indicator is changed due to contact with the feces.

According to the sensor unit, the sensor unit is easily attached at positions corresponding to the indicators.

In such a sensor unit, a color of the mark may be different from a color of the urine indicator after the urine indicator is changed due to contact with the urine, and the color of the mark may be different from a color of the feces indicator after the feces indicator is changed due to contact with the feces.

According to the sensor unit, by making the color of the mark different from the colors of the indicators after the change, it reduces a risk that the change in the color of each indicator is difficult to be recognized when the indicator is directly viewed.

There is also provided an absorbent article kit including: an absorbent article; and a sensor unit, the absorbent article including: a urine indicator that visually changes a color due to contact with urine and that is formed extending along a vertical direction of the absorbent article; and a feces indicator that visually changes a color due to contact with feces and that is formed extending along the vertical direction of the absorbent article, the sensor unit including: a urine change-detection unit that detects a change in color of the urine indicator; and a feces change-detection unit that detects a change in color of the feces indicator.

According to the absorbent article kit, it makes it easier to attach the sensor unit to the absorbent article and makes it easier to recognize whether the wearer has excreted urine or the wearer has excreted feces.

In such an absorbent article kit, the sensor unit has an attachment side to be attached to the absorbent article, in a state where the sensor unit is attached to the absorbent article while the attachment side facing the urine indicator and the feces indicator, a vertical length of the sensor unit may be shorter than a vertical length of the urine indicator, and the vertical length of the sensor unit may be shorter than a vertical length of the feces indicator.

According to the absorbent article kit, the urine indicator and the feces indicator can be directly visually recognized, making it possible to confirm excrement of the wearer together with the detection results obtained by the sensor unit.

In such an absorbent article kit, the sensor unit may have flexibility, and stiffness of a portion of the sensor unit in which the urine change-detection unit and the feces change-detection unit are not provided may be lower than stiffness of a vertical central portion and a lateral central portion of the absorbent article.

According to the absorbent article kit, it is possible to enhance the texture during usage of the absorbent article.

There is also provided an absorbent-article packaged piece (or absorbent-article package) including: a packaging member (or packaging medium); a plurality of absorbent articles; and a sensor unit, the plurality of absorbent articles and the sensor unit being accommodated in the packaging member, a number of the sensor unit being smaller than a number of the absorbent articles, each of the absorbent articles including: a urine indicator that visually changes a color due to contact with urine and that is formed extending along a vertical direction of the absorbent article; and a feces indicator that visually changes a color due to contact with feces and that is formed extending along the vertical direction of the absorbent article, the sensor unit including: a urine change-detection unit that detects a change in color of the urine indicator; and a feces change-detection unit that detects a change in color of the feces indicator.

According to the absorbent-article packaged piece, since one sensor unit can be attached to a plurality of absorbent articles, it is possible to reduce the cost of the absorbent-article packaged piece.

The following describes an example of a tape-type disposable diaper for infants as an absorbent article according to one or more embodiments of the present invention.

However, there is no limitation thereto. The present invention is also applicable to, for example, absorbent articles as follow: a pull-on disposable diaper, a pad-type disposable diaper, a tape-type disposable diaper for adults, and the like.

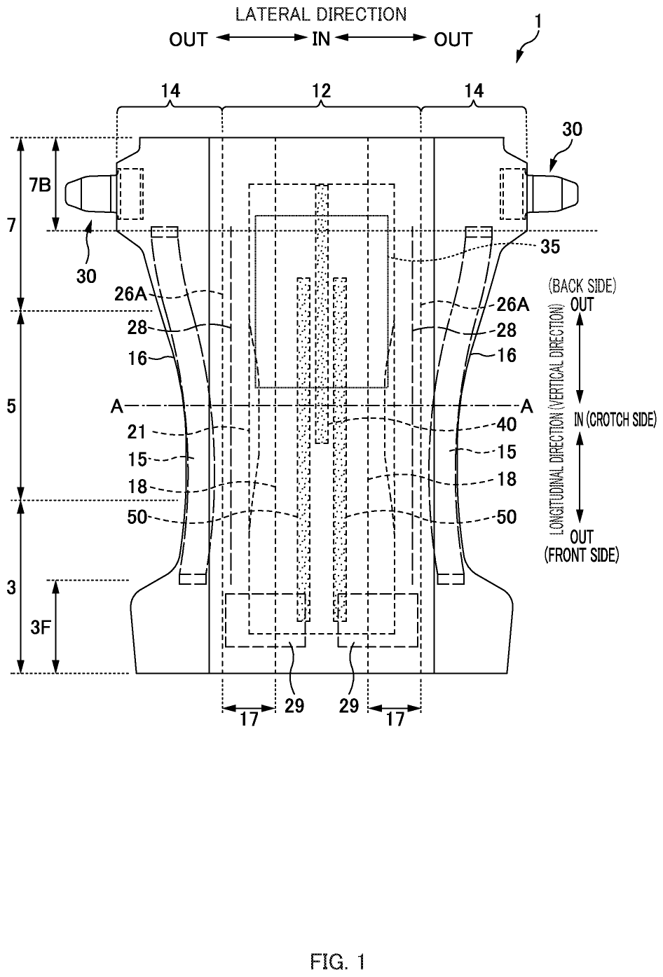

Basic Configuration of Tape-Type Disposable Diaper 1 FIG. 1 is a plan view of a tape-type disposable diaper 1 (hereinafter, also referred to as a “diaper 1”) in an unfolded and stretched state when viewed from a skin side.

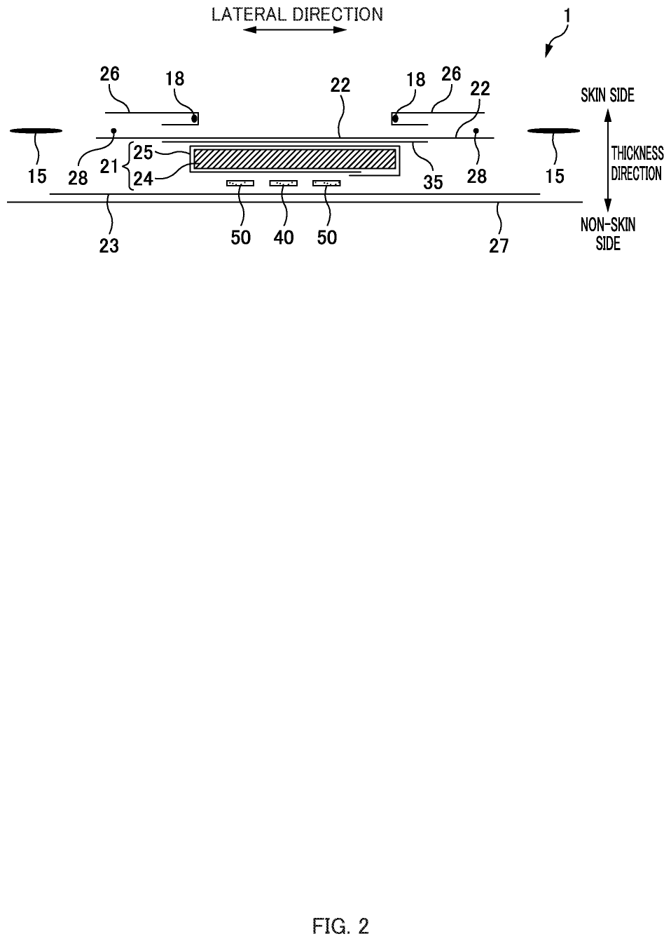

FIG. 2 is a cross-sectional view taken along a line A-A of FIG. 1.

The unfolded state of the diaper 1 refers to a state in which the diaper 1 is unfolded on a plane by opening the diaper 1 in the longitudinal direction.

In addition, the stretched state of the diaper 1 refers to a state where the diaper 1 has been unfolded and stretched such that wrinkles formed in the diaper 1 are substantially no longer visible, that is to say a state where the diaper 1 has been stretched until the dimensions of constituent members of the diaper 1 (e.g., a later-described top sheet 22) match or are close to the dimensions of the members on their own.

The diaper 1 of one or more embodiments is a so-called open-type disposable diaper and has a front portion 3, a crotch portion 5, and a back portion 7 in the longitudinal direction as shown in FIG. 1.

The front portion 3 is a portion that is located at the wearer's front portion (front side) while the diaper 1 is put on.

The back portion 7 is a portion that is located at the wearer's back portion (back side) while the diaper 1 is put on.

The crotch portion 5 is provided between the front portion 3 and the back portion 7.

As shown in FIG. 1, the directions used in the following description are defined as follows.

That is, in an unfolded state as viewed from above, the direction from the front portion 3 toward the back portion 7 is defined as the “longitudinal direction” and the direction orthogonal to the longitudinal direction is defined as the “lateral direction”.

The line A-A shown in FIG. 1 indicates the center of the diaper 1 in the longitudinal direction.

Further, as shown in FIG. 2, the direction orthogonal to the longitudinal direction and the lateral direction is defined as the “thickness direction”, the side facing the wearer's skin is defined as the “skin side”, and a side opposite to the skin side is defined as the “non-skin side”.

The “longitudinal direction” in the unfolded state is a direction along the “vertical direction” in a state where the diaper 1 is put on.

The diaper 1 includes a central band-shaped region 12, side flaps 14, leg gathers 16, and leg side gathers 17.

Fastening tapes 30 are respectively attached to the back portion 7 of the pair of side flaps 14.

The central band-shaped region 12 is a band-shaped region that is located in the central portion in the lateral direction and that is constituted by the front portion 3, the crotch portion 5, and the back portion 7 (see FIG. 1).

The central band-shaped region 12 is a portion that absorbs and holds a liquid such as urine that is excreted by the wearer.

The central band-shaped region 12 has a longitudinally-elongated shape (shape extending along the longitudinal direction) that includes a liquid-retaining absorbent body 21.

The central band-shaped region 12 mainly includes: the absorbent body 21; a liquid-permeable top sheet 22 that covers the liquid-absorbent absorbent body 21 from the skin side; a liquid-impermeable back sheet 23 that covers the absorbent body 21 from the non-skin side; and an exterior sheet 27 that forms the exterior of the diaper 1 (e.g., a nonwoven fabric) (see FIG. 2).

In the central band-shaped region 12, a liquid-permeable second sheet 35 is further provided.

However, it is not necessarily required that the second sheet 35 is provided.

As shown in FIG. 2, the absorbent body 21 of one or more embodiments includes an absorbent core 24 that absorbs excrement such as urine, and a liquid-permeable core-wrapping sheet 25 that covers the absorbent core 24 from both the skin side and the non-skin side in the thickness direction.

Examples of suitable materials for the core-wrapping sheet 25 include a tissue paper, a nonwoven fabric, and the like.

However, the core-wrapping sheet 25 is not essential.

The absorbent body 21 is arranged extending over the front portion 3, the crotch portion 5, and the back portion 7.

The absorbent core 24 of one or more embodiments has a substantially hourglass shape in a plan view as an example of a predetermined shape.

The liquid-absorbent material that constitutes the absorbent core 24 can be made of liquid-absorbent fibers (e.g., pulp fibers) or liquid-absorbent granules (e.g., a superabsorbent polymer, so-called SAP), for example.

The liquid-absorbent material may also include a liquid-absorbent material other than the liquid-absorbent fibers and the liquid-absorbent granules.

The side flaps 14 are portions located on two lateral side portions of the central band-shaped region 12.

The side flaps 14 are formed extending over the front portion 3, the crotch portion 5, and the back portion 7 (see FIG. 1).

The lateral length (width) of the side flaps 14 in the crotch portion 5 is smaller than the lateral length (width) of the side flaps 14 in the front portion 3 and the back portion 7.

The side flaps 14 are each mainly constituted by skin-side sheets 26 and the back sheet 23 (see FIG. 2).

The skin-side sheets 26 are skin-side members that are formed extending over the front portion 3, the crotch portion 5, and the back portion 7, and are constituted by a nonwoven fabric, for example.

The skin-side sheets 26 are members that constitute the leg side gathers 17 (barrier cuffs), and the outer portions of the skin-side sheets 26 (portions outside of joining portions 26A indicated by dashed lines in FIG. 1) constitute the side flaps 14.

In the central band-shaped region 12, at least in the crotch portion 5 are provided with a pair of leg elastic members 28 (e.g., elastic strings) that are capable of stretching and contracting in the longitudinal direction, between the skin-side sheets 26 and the top sheet 22.

The leg elastic members 28 are members that give stretchability to the central band-shaped region 12 in the crotch portion 5, and are attached in a state of being stretched in the longitudinal direction.

Accordingly, the leg elastic members 28 apply a contractive force acting in the longitudinal direction to the crotch portion 5 of the central band-shaped region 12.

The pair of side flaps 14 are each provided with a leg-gather elastic member 15 that stretches and contracts in the longitudinal direction.

The leg-gather elastic members 15 are elastic members that stretch and contract in the longitudinal direction, and are members that give stretchability to the leg openings while the diaper 1 is put on.

Specifically, the leg-gather elastic members 15 are leg elastic members that allow the leg portion of the diaper 1 to fit to the legs of the wearer.

The leg-gather elastic members 15 may be, for example, a band-shaped elastic sheet having stretchability, a plurality of elastic strings, or the like.

The leg-gather elastic members 15 give stretchability to the skin-side sheets 26 and the back sheet 23 of the crotch portion 5, thereby constituting the leg gathers 16.

The leg side gathers 17 are barrier cuffs for preventing the leakage of liquids through gaps around the legs.

The pair of leg side gathers 17 extend along the longitudinal direction over the front portion 3, the crotch portion 5, and the back portion 7 (see FIG. 1).

The leg side gathers 17 are formed inside of the side flaps 14 so as to cover the two edges of the central band-shaped region 12.

The leg side gathers 17 (barrier cuffs) are mainly constituted by portions of the skin-side sheet 26 that are inside in the lateral direction (see FIG. 2).

The inner edges of the skin-side sheets 26 in the crotch portion 5 have stretchability due to a leg-side-gather elastic members 18 such as elastic strings.

The skin-side sheets 26 are each joined along the longitudinal direction in the joining portion 26A that is between the central band-shaped region 12 and the side flap 14.

While the diaper 1 is put on, due to the stretchability of the leg-side-gather elastic member 18, the region located laterally inside the joining portion 26A in the skin-side sheet 26 rises toward the wearer's skin at the joining portion 26A, thereby suppressing lateral leakage of excrement or the like.

The back portion 7 has a back waist portion 7B positioned on the wearer's back waist while the diaper is put on (see FIG. 1).

The back waist portion 7B is a portion extending from the longitudinal outer end of the back portion 7 to the longitudinal inner end of portions where the side flaps 14 each extend in the lateral direction.

The fastening tapes 30 are arranged on two lateral side portions of the portions where the side flaps 14 each extend in the lateral direction, in the back waist portion 7B of the diaper 1 (see FIG. 1).

Further, by engaging the fastening tapes 30 with later-described target tapes 29 (FIG. 1), a waist opening and a leg opening of the diaper 1 are formed, making it possible to fix the position of the diaper 1 with respect to the wearer's body (trunk).

The front portion 3 has a front waist portion 3F positioned on the wearer's front waist while the diaper is put on (see FIG. 1).

The front waist portion 3F is a region corresponding to the above-described back waist portion 7B while the diaper is put on.

The target tapes 29 are provided in the front waist portion 3F of the central band-shaped region 12.

The target tapes 29 are each arranged on the non-skin side of the exterior sheet 27 of the front portion 3.

The target tapes 29 are members that can respectively engage with the fastening tapes 30, and are formed by nonwoven fabric, for example.

The target tapes 29 constitute a target region for engagement with the fastening tapes 30.

It should be noted that, instead of the target tapes 29 being arranged on the non-skin side of the exterior sheet 27, the target region may be directly formed on the outermost piece of nonwoven fabric that constitutes the exterior sheet 27.

Also, the diaper 1 is put on by engaging the fastening tapes 30 with the target tapes 29.

As shown in FIGS. 1 and 2, the diaper 1 of one or more embodiments includes a feces indicator 40 and urine indicators 50 on the non-skin side in the thickness direction with respect to the absorbent body 21, the feces indicator 40 being an indicator that visually changes a color due to contact with feces, the urine indicators 50 each being an indicator that visually changes a color due to contact with urine.

The feces indicator 40 and the urine indicators 50 are separated from each other in the lateral direction and are arranged extending along the longitudinal direction, in a band-like shape. But the feces indicator 40 and the urine indicators 50 does not have to be separated in the lateral direction.

In addition, the indicators may be arranged discontinuously in the longitudinal direction.

The urine indicators 50 are indicators including a pH indicator which is employed in a conventional common diaper.

For example, in the urine indicators 50, the pH of urine is used as a reaction factor (urine-indicator reaction factor), and the discharge of urine is detected when the urine indicators 50 exhibit a predetermined reaction (e.g., color reaction) upon contact with urine.

The feces indicator 40 will be described later in detail.

Principle of Feces Indicator 40

The feces indicator 40 is a feces indicator for an absorbent article such as the diaper 1, and detects the excretion of feces by exhibiting a predetermined reaction (e.g., color reaction) upon contact with a predetermined reaction factor (feces-indicator reaction factor) that is contained in feces.

In one or more embodiments, the feces indicator 40 includes a chemical component that detects a biological substance in feces, and the response of the chemical component to feces is different from the response to urine, making it possible to detect only the excretion of feces.

For example, in the case where a protein is a target biological substance (feces-indicator reaction factor) to be detected by the chemical component that is contained in the feces indicator 40, it is possible to use a pH indicator as the chemical component.

Generally, a protein has a structure in which amino acids are polymerized, and has acidic and basic functional groups at both ends of the main chain and the side chains of the protein. Therefore, in the case where a certain amount or more of the protein is present, it is possible to make the pH indicator change its color (protein error method).

In one or more embodiments, the pH indicator responds to feces by detecting undigested protein derived from food in feces, protein secreted from intestinal bacteria, or the like.

As a specific pH indicator, for example, it is possible to use tetraphenol blue.

In this case, if the protein is present, the pH indicator binds to a free amino group in the protein to form a salt-like blue compound, and exhibits a blue color corresponding to a pH that is higher than the true pH.

Accordingly, when the feces indicator 40 including tetraphenol blue comes into contact with feces, the feces indicator changes color from yellow to blue.

It should be noted that, in order to make it easier to change the color of the pH indicator, the pH may be set in advance to the acidic side at approximately pH 3.

Therefore, the pH indicator may include a citrate buffer or the like.

In this manner, by using a predetermined pH indicator, it is possible to make the pH indicator change color in response to a protein, without the occurrence of color change caused by a change in the pH of urine or feces itself.

It should be noted that the pH indicator used in the protein error method is not limited to the above-described tetraphenol blue, and it is possible to use other pH indicators.

For example, it is possible to use bromophenol blue, bromocresol green, thymolphthalein, and the like, or other indicators.

Furthermore, the pH indicator may be safe for the skin and may have an excellent storage properties with respect to moisture or sunlight.

Further, in the feces indicator 40, the chemical component included in the feces indicator 40 may respond to feces and may not respond to urine so as not to erroneously detect whether the excrement is feces or urine.

Therefore, the feces indicator 40 in one or more embodiments exhibits a reaction such as a color reaction in response to a feces-indicator reaction factor (protein or the like) which has a predetermined concentration or higher. In the case where the concentration of the feces-indicator reaction factor is lower than the predetermined concentration, it is less likely to cause the reaction.

Generally, protein is not included in the urine of a healthy person, and even in the case of a non-healthy person, the amount of protein in the urine is less than 10,000 mg/L.

Therefore, in one or more embodiments, with the protein error method using a pH indicator, the feces indicator 40 responds to preferably 150 mg/L or more of protein, responds to more preferably 5,000 mg/L or more of protein, and responds to even more preferably 10,000 mg/L or more of protein.

For example, under the condition where bromophenol blue is used as the chemical component, in the case where the feces indicator 40 responds to 150 mg/L or more of protein, the amount of the pH indicator applied per 1 cm² in the feces indicator 40 may be 16.3 μg, in the case where the feces indicator 40 responds to 5,000 mg/L or more of protein, the amount of the pH indicator applied per 1 cm² in the feces indicator 40 may be 0.5 μg, and in the case where the feces indicator 40 responds to 10,000 mg/L or more of protein, the amount of the pH indicator applied per 1 cm² in the feces indicator 40 may be 0.25 μg.

It should be noted that setting the amount of the pH indicator applied to 17.0 μg or less enhances the safety of the feces indicator 40 for the wearer of the absorbent article.

In one or more embodiments, by adjusting the amount of the pH indicator applied as described above, the range in which the feces indicator 40 can react to urine can be made relatively narrower compared with the range in which the feces indicator 40 can react to feces.

In other words, the reaction of the feces indicator 40 to feces can be made different from the reaction of the feces indicator 40 to urine.

This enables to make the feces indicator 40 difficult to react to urine.

Further, a reaction factor of the feces indicator 40 is not limited to the protein described above.

For example, it is acceptable that the feces indicator 40 reacts to the intestinal bacteria contained in the feces, the ionic strength of feces that correlates with the specific gravity of the feces, and a feces-derived substance such as bilirubin, which is a bile pigment.

These components are generally not included in urine or are contained in a very small amount or a very small specific gravity in urine compared with feces.

Accordingly, in the same manner as in the case of using a protein as a reaction factor, the feces indicator 40 is less likely to react to urine and is more likely to react to feces.

Therefore, it is possible to detect the feces excreted in the diaper 1 with high accuracy.

Specific Configuration of Feces Indicator 40

The feces indicator 40 is formed by applying an adhesive (e.g., hot-melt adhesive HMA) containing the chemical component (e.g., pH indicator) described above to the skin-side surface of the back sheet 23 of the diaper 1.

In one or more embodiments, as shown in FIG. 1, the feces indicator 40 is formed by applying a hot-melt adhesive (HMA) using a coater to a strip-shaped (or linear) region which extends along the longitudinal direction over the front portion 3, the crotch portion 5, and the back portion 7.

According to the above-described coater application, it is possible to form the feces indicator 40 whose film thickness is uniform and that has little unevenness, making it possible to enhance the detection accuracy of feces.

Further, it is possible to reduce manufacturing costs.

It should be noted that it is possible to form the urine indicator 50 in the same manner.

Alternatively, the feces indicator 40 may be formed by mixing a chemical component with ink and performing print coating on the back sheet 23 or the core-wrapping sheet 25 with the mixture.

Alternatively, the feces indicator 40 may be formed as follow: a filter paper or a nonwoven fabric impregnated with the chemical component is joined and fixed to the back sheet 23 or the core-wrapping sheet 25 with a hot-melt adhesive (HMA) or by ultrasonic welding.

Color Reaction of Feces Indicator 40 and Urine Indicator 50

As shown in FIG. 1, in one or more embodiments, the feces indicator 40 and the urine indicators 50 are provided in a strip-like shape extending along the longitudinal direction while being spaced apart from each other in the width direction, the urine indicators 50 being located on two lateral sides of the feces indicator 40.

The feces indicator 40 is an indicator that visually changes a color due to contact with feces (undergoes a so-called color reaction). The feces indicator 40 has a pre-change color which is a color before the color change and a post-change color which is a color after the color change due to contact with feces.

The urine indicators 50 are each an indicator that visually changes a color due to contact with urine (undergoes a so-called color reaction).

The urine indicator 50 similarly has a pre-change color which is a color before the color change and a post-change color which is a color after the color change due to contact with urine.

In one or more embodiments, the chemical component included in each of the indicators 40 and 50 is adjusted such that the colors after the coloration are different from each other.

For example, in the case where tetraphenol blue is used as the chemical component (pH indicator) of the feces indicator 40, the color after reaction with feces is “blue”.

In this case, as the chemical component of the urine indicator 50, chemical component whose color after reaction with urine is changed to a color different from blue (e.g., green) is used, forming the urine indicator 50.

In a case where the pre-change color and the post-change color of each of the feces indicator 40 and the urine indicator 50 are specified as the closest hues among the 24 color hues of the Ostwald color wheel, the expression that the colors are “identical” or “similar” refers to a state where the hues are identical in the Ostwald color wheel, and the expression that the colors are “different” refers to a state where the hues are shifted by one or more color numbers in the color space of the Ostwald color system from each other.

In one or more embodiments, the pre-change color and the post-change color of each of the feces indicator 40 and the urine indicators 50 have the following configurations.

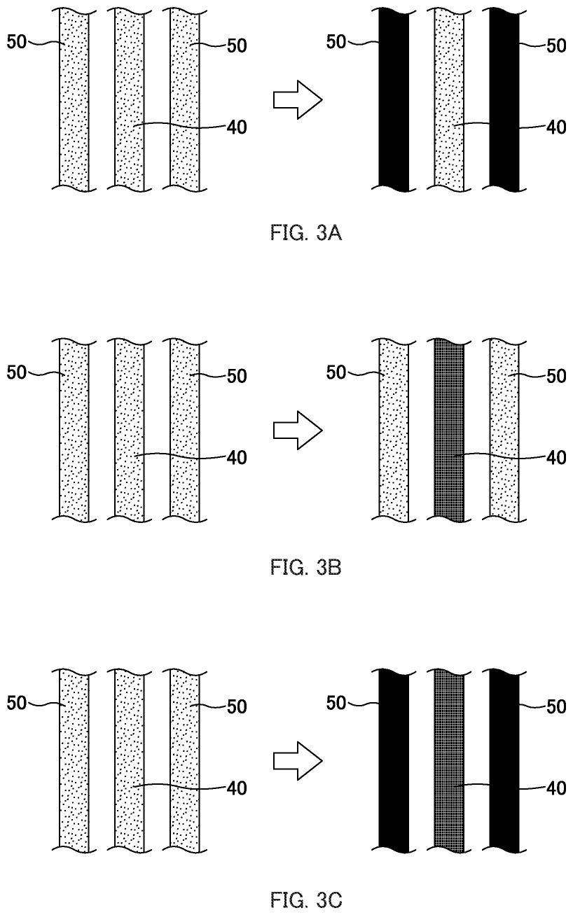

FIGS. 3A, 3B, and 3C each show a state of parts of the feces indicator 40 and the urine indicators 50 in FIG. 1 viewed from the non-skin side of the diaper 1.

The left diagram of FIG. 3A shows a state before the colors of the feces indicator 40 and the urine indicators 50 are changed (that is, before use).

As shown in the drawing, in one or more embodiments, the pre-change color of the feces indicator 40 (e.g., white) and the pre-change color of the urine indicators 50 (e.g., white) are identical or similar colors.

In the right diagram of FIG. 3A, it can be found that the urine indicators 50 changes the color in response to urine (e.g., green), which results in the feces indicator 40 and the urine indicators 50 that had identical or similar colors being made have different colors.

A person replacing the diaper 1 seeing this can recognize that urination has been occurred.

In FIG. 3B, before use, the pre-change colors of the feces indicator 40 and the urine indicators 50 are identical or similar colors (e.g., white) (left diagram of FIG. 3B). In the right diagram of FIG. 3B, it can be found that the feces indicator 40 changes a color in response to feces (e.g., blue), which results in the feces indicator 40 and the urine indicator 50 being made have different colors.

The person replacing the diaper 1 seeing this can recognize that defecation has been occurred.

In FIG. 3C, the pre-change colors of the feces indicator 40 and the urine indicators 50 are identical or similar colors (e.g., white) (the left diagram of FIG. 3C). But, the feces indicator 40 and the urine indicators 50 respectively detect defecation and urination, and in the right diagram of FIG. 3C, the indicators (40, 50) change their colors from the pre-change color.

The post-change colors of the feces indicator 40 and the urine indicators 50 (e.g., the post-change color of the feces indicator 40 is blue, and the post-change color of the urine indicators is green) are dissimilar colors.

The person replacing the diaper 1 seeing this can recognize that both defecation and urination have been occurred.

In one or more embodiments, the pre-change colors of the feces indicator 40 and the urine indicator 50 are set to white that is an identical color or a similar color to the color of the diaper 1, but the present invention is not limited thereto.

The pre-change colors of the feces indicator 40 and the urine indicator 50 may be set to be different (e.g., red) from an identical color or a similar color to the color of the diaper 1.

In addition, the post-change colors of the feces indicator 40 and the urine indicator 50 may be each set to any of the primary colors.

By setting the post-change colors to the primary color, it is possible to more accurately detect the post-change colors by a later-described sensor unit 80.

Sensor Unit 80

The sensor unit 80 is a unit for an absorbent article, is a separate component from the diaper 1, and is used by being attached to the non-skin-side surface of the diaper 1.

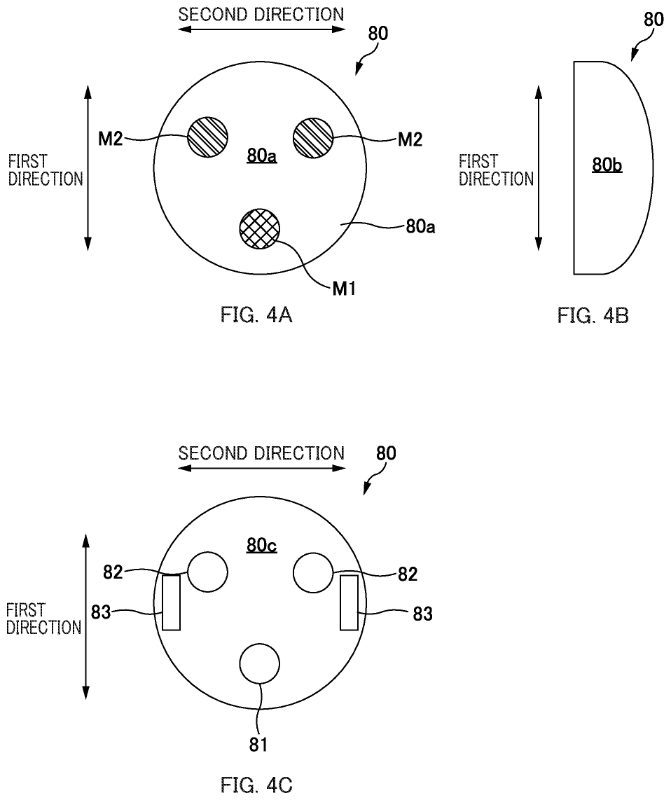

FIG. 4A is a top view of the sensor unit 80.

FIG. 4B is a side view of the sensor unit 80.

FIG. 4C is a bottom view of the sensor unit 80.

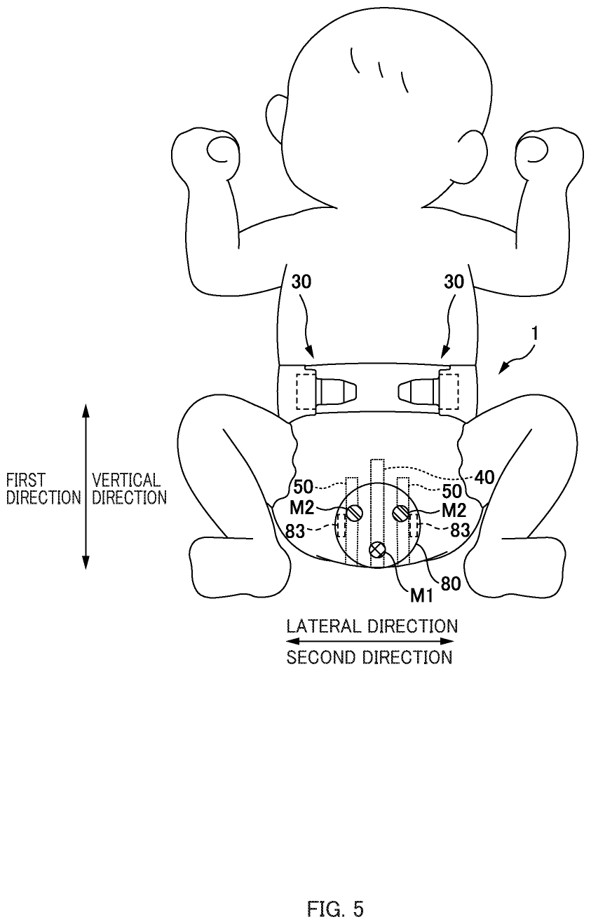

FIG. 5 is a diagram illustrating an example of how the sensor unit 80 is attached.

It should be noted that the absorbent article (e.g., the diaper 1) and the sensor unit 80 will be collectively referred to as an absorbent article kit (diaper kit).

The sensor unit 80 of one or more embodiments has, for example, a light blue, plastic exterior body.

The sensor unit 80 may have a light color such as light blue.

This makes it easier to give an impression that the sensor unit 80 attached to the diaper 1 has a soft atmosphere, to the person replacing the diaper 1.

In addition, in the case where the post-change colors of the feces indicator 40 and the urine indicator 50 is set to any of the primary colors, it makes the person replacing the diaper 1 easier to recognize the change in the post-change colors of the feces indicator 40 and the urine indicator 50 adjacent to the light-colored sensor unit 80.

As shown in FIG. 4A, the upper surface 80a of the sensor unit 80 has a circular outer shape.

As shown in FIG. 4B, the side surface 80b of the sensor unit 80 has a shape in which a substantially rectangular surface and a curved surface are combined.

As shown in FIG. 4C, the bottom face 80c of the sensor unit 80 is a surface on the side where the substantially rectangular surface of the side surface 80b is provided and on the side to be attached to the diaper 1.

The bottom face 80c has a circular shape identical to the shape of the upper surface 80a, and includes a transparent portion 81, two transparent portions 82, and two attaching/detaching mechanisms 83.

The transparent portion 81 and the transparent portions 82 are portions where circular transparent glass or plastic is provided. Through the transparent portion 81 and the transparent portions 82, it is possible to visually recognize the inside of the sensor unit 80.

The attaching/detaching mechanisms 83 each are an attaching/detaching mechanism that makes the sensor unit 80 possible to be attached to or detached from the non-skin-side surface (exterior sheet 27) of the diaper 1, that is, for example a hook-and-loop fastener.

As shown in FIG. 5, by pressing the sensor unit 80 to the diaper 1 so that the bottom face 80c of the sensor unit 80 faces the exterior sheet 27 of the diaper 1, the attaching/detaching mechanisms 83 are fixed to the exterior sheet 27, and the sensor unit 80 becomes in a state of being attached to the diaper 1.

It should be noted that, when the sensor unit 80 is pulled so as to be peeled off from the diaper 1, the sensor unit 80 can be detached from the diaper 1.

When viewed from the bottom face 80c side, in the case where the transparent portion 81 and the transparent portions 82 overlap the attaching/detaching mechanisms 83, the attaching/detaching mechanisms 83 cover the transparent portion 81 and the transparent portion 82, causing a risk of hindering the detection of the change in each color of the feces indicator 40 and the urine indicator 50.

Therefore, the transparent portion 81 and the transparent portions 82 may have portions that do not overlap the attaching/detaching mechanisms 83, and further, the transparent portion 81, the transparent portions 82, and the attaching/detaching mechanisms 83 may not overlap in any way.

Thus, it is possible to reduce the risk of hindering the detection of the change in each color of the feces indicator 40 and the urine indicator 50 by the attaching/detaching mechanisms 83.

In addition, similarly, a later-described feces change-detection unit 812r and later-described urine change-detection units 822r may not overlap the attaching/detaching mechanisms 83 when viewed from the bottom face 80c.

In the case where the entire region of the feces change-detection unit 812r or the entire region of the urine change-detection unit 822r overlaps the attaching/detaching mechanism 83, it hinders the detection of the change in the colors of the feces indicator 40 and the urine indicators 50. The configuration makes it possible to reduce such a risk.

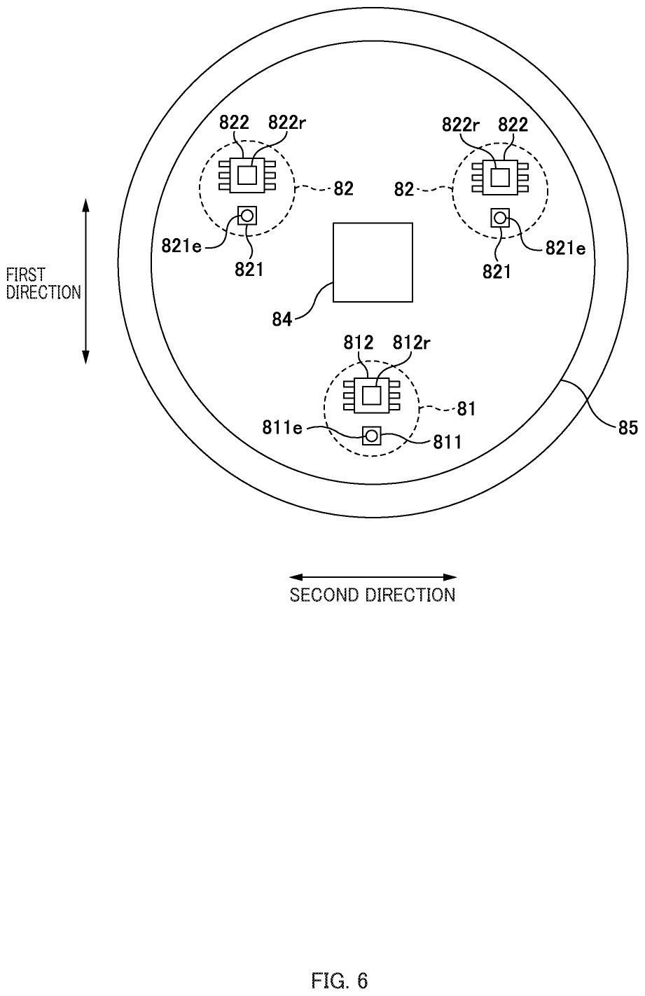

FIG. 6 is a schematic explanatory diagram illustrating the inside of the sensor unit 80 when viewed from the bottom face side.

FIG. 6 illustrates a state of removing the bottom face 80c (including the transparent portion 81 and the transparent portions 82) of the sensor unit 80.

The sensor unit 80 includes inside: a light emitting device 811; a light emitting device 821; a feces change-detection device 812; a urine change-detection device 822; a control unit (or controller) 84; and a substrate 85.

It should be noted that, as shown in FIG. 6, when the sensor unit 80 is viewed from the bottom face side, the light emitting device 811 and the feces change-detection device 812 are provided at positions that overlap the transparent portion 81 in the thickness direction, and the light emitting device 821 and the urine change-detection device 822 are provided at positions that overlap the transparent portion 82 in the thickness direction.

It should be noted that FIG. 6 shows a state of removing the transparent portion 81 and the transparent portions 82, and, for convenience, the positions corresponding to the transparent portion 81 and the transparent portions 82 are indicated by dotted lines.

The substrate 85 is provided for mounting electronic components such as the light emitting devices 811 and 821 and the detection devices 812 and 822.

The control unit 84 transfers data to and from each of the devices 811, 821, 812, and 822 and controls the sensor unit 80. The control unit 84 is realized by a central processing unit (CPU) executing a program stored in a predetermined memory.

The control unit 84 includes (or functions as): an instructing unit (not shown) that instructs the light emitting devices 811 and 821 to emit light; a receiving unit (not shown) that receives a result detected by each of the detection devices 812 and 822; and an output unit (not shown) that outputs the result detected by each of the detection devices 812 and 822.

The light emitting device 811 includes a light source 811e that emits light to a measurement surface.

The light emitting devices 821 each include a light source 821e that emits light to the measurement surface.

The light sources 811e and 821e are, for example, light emitting components such as LEDs.

The feces change-detection device 812 includes the feces change-detection unit 812r that comprises color sensing elements and detects the color of the measurement surface by receiving the light reflected on the measurement surface from the light source 811e.

The urine change-detection devices 822 each include the urine change-detection unit 822r that comprises color sensing elements and detects the color of the measurement surface by receiving the light reflected on the measurement surface from the light source 821e.

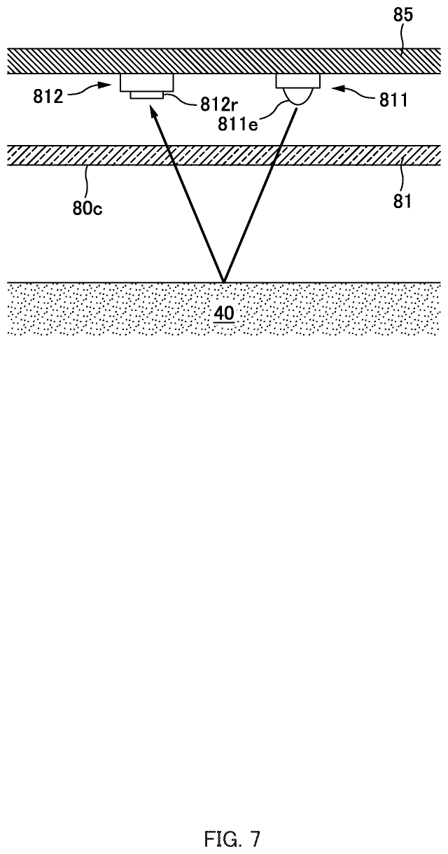

FIG. 7 is an explanatory diagram illustrating the principle of measuring the color of the feces indicator 40 by the sensor unit 80.

FIG. 7 illustrates a state where the sensor unit 80 is arranged above the feces indicator 40.

The principle of measuring the color of the feces indicator 40 will be described using FIGS. 5 and 7, and the principle of measuring the color of the urine indicator 50 is also the same.

In one or more embodiments, the light sources 811e and 821e project three LED light beams (white light) of red (R), green (G), and blue (B) onto the measurement surface.

The feces change-detection device 812 and the urine change-detection devices 822 are sensors that can distinguish colors digitally, and are so-called RGB color sensors.

The feces change-detection device 812 and the urine change-detection devices 822 detect a ratio of the light amounts of the respective color components of RGB to be reflected from the measurement surface.

The ratio of the colors of red, green, and blue to be reflected varies depending on the color of the measurement surface, and therefore the color of the measurement surface is determined from the ratio of the respective colors.

First, as shown in FIG. 5, the sensor unit 80 is attached to the diaper 1 by being attached to the exterior sheet 27, which is the non-skin-side surface of the diaper 1, using the attaching/detaching mechanisms 83 such that the transparent portion 81 and at least a part of the feces indicator 40 face each other and such that the transparent portions 82 and at least a part of the urine indicators 50 face each other.

In one or more embodiments, the sensor unit 80 is attached to the wearer's front side.

In a state where the sensor unit 80 is attached to the diaper 1, it may decrease a portion where the attaching/detaching mechanisms 83 overlaps any of the feces indicator 40 and the urine indicators 50 when viewed from the upper surface 80a side or from the bottom face 80c side.

This is for reducing the risk that covering the feces indicator 40 and the urine indicators 50 by the attaching/detaching mechanisms 83 makes it impossible to accurately detect a change in color.

According to the instruction from the instructing unit of the control unit 84, the light emitting device 811 causes light to be emitted from the light source 811e toward the feces indicator 40 through the transparent portion 81.

Then, the feces change-detection unit 812r receives the light reflected on the surface of the feces indicator 40 through the transparent portion 81.

Since the ratio of the colors of red, blue, and green of the received light varies depending on the color of the feces indicator 40, the color is distinguished by detecting the ratio of the received light.

For example, in the case where the color of the feces indicator 40 is white which is the pre-change color, all of the red light, the green light, and the blue light are more likely to be reflected.

Therefore, the feces change-detection unit 812r detects light having a ratio of red, green, and blue of 1:1:1.

Based on this ratio, the feces change-detection device 812 determines that the color of the feces indicator 40 is white.

On the other hand, in the case where the color of the feces indicator 40 is blue which is the post-change color, the blue light is more likely to be reflected, but the red light and the green light are less likely to be reflected.

Therefore, the feces change-detection unit 812r detects light having a ratio of red, green, and blue of 1:1:4.

Based on this ratio, the feces change-detection device 812 determines that the color of the feces indicator 40 is blue.

The output unit outputs the determined color of the measurement surface to a terminal (e.g., a tablet, a smartphone, or a computer) in which wireless communication is set in advance and which is owned by the person replacing the diaper 1 or the like.

The output unit is set to output a result in the case where blue color is detected on the measurement surface in advance by the feces change-detection device 812, and a result in the case where green color is detected on the measurement surface by the urine change-detection device 822.

The terminal to which an output is sent from the output unit displays on a screen that the color of the feces indicator 40 is changed or the color of the urine indicator 50 is changed.

By outputting the measurement result wirelessly, it eliminates a risk that the person replacing the diaper 1 is bothered by effort and trouble of wiring which is caused in the case of wired line.

In addition, the sensor unit 80 is easily attached to the diaper 1.

The person replacing the diaper 1 can recognize the received measurement result, can visually recognize the exhibition of the change in the color(s) of the feces indicator 40 and/or the urine indicators 50 of the diaper 1, and can recognize whether defecation has occurred or urination has occurred.

Since the person replacing the diaper 1 can recognize the state of defecation and urination by confirming the terminal or the like, the person replacing the diaper 1 can confirm the excretion state even when the wearer is wearing clothes.

In the state where the sensor unit 80 is attached to the diaper 1, a direction extending along the vertical direction of the diaper 1 is defined as a “first direction” of the sensor unit 80, and a direction extending along the lateral direction of the diaper 1 is defined as a “second direction”. With respect to the first direction, compared with the case where the position of the center of the feces change-detection unit 812r is identical to the positions of the centers of the urine change-detection unit 822r, the position of the center of the feces change-detection unit 812r may be different from the positions of the centers of the urine change-detection units 822r.

The first direction and the second direction of the sensor unit 80 shown in FIG. 6 are directions respectively corresponding to the first direction and the second direction in a state shown in FIG. 5 where the sensor unit 80 is attached to the diaper 1.

In other words, in the attached state, the position of the center of the feces change-detection unit 812r in the vertical direction and the positions of the centers of the urine change-detection units 822r in the vertical direction may be different from each other.

Since, in the first direction, the position of the center of the feces change-detection unit 812r and the positions of the centers of the urine change-detection units 822r are different from each other, the position of the center of the transparent portion 81 and the positions of the centers of the transparent portions 82 may be different in the first direction from each other.

Therefore, compared with the case where the position of the center of the feces change-detection unit 812r and the positions of the centers of the urine change-detection units 822r are provided at the same position in the first direction (vertical direction), it is possible to easily detect the change in the color of the feces indicator 40 and the change in the colors of the urine indicators 50 at different positions in the vertical direction.

For example, due to the structure of the body, feces are more likely to be excreted on the lower side with respect to urine. Accordingly, in the case where the sensor unit 80 is attached to the front side (stomach-side) of the diaper 1, the change in the color of the feces indicator 40 is more likely to exhibit on the lower side than the change in the color of the urine indicator 50 is.

Therefore, as shown in FIGS. 5 and 6, in the sensor unit 80, the feces change-detection unit 812r is provided on the lower side with respect to the urine change-detection units 822r, making it easier to more accurately detect the change in the color of the feces indicator 40 and the change in the colors of the urine indicators 50.

In addition, in the state where the sensor unit 80 is attached to the diaper 1, with respect to the second direction, compared with the case where the position of the center of the feces change-detection unit 812r is identical to the positions of the centers of the urine change-detection units 822r, the position of the center of the feces change-detection unit 812r may be different from the positions of the centers of the urine change-detection units 822r.

That is, in the attached state, the lateral position of the center of the feces change-detection unit 812r and the lateral positions of the centers of the urine change-detection units 822r may be different from each other.

Since, in the second direction, the position of the center of the feces change-detection unit 812r and the positions of the centers of the urine change-detection units 822r are different from each other, the position of the center of the transparent portion 81 and the positions of the centers of the transparent portions 82 may be different in the second direction from each other.

Therefore, compared with the case where the position of the center of the feces change-detection unit 812r and the positions of the centers of the urine change-detection units 822r are provided at the same position in the second direction (lateral direction), it is possible to easily detect the change in the color of the feces indicator 40 and the change in the colors of the urine indicators 50 at different positions in the lateral direction.

Further, the urine change-detection units 822r and 822r may be provided on two sides of the feces change-detection unit 812r in the second direction (lateral direction).

As in one or more embodiments, even in the case where the sensor unit 80 is attached to the diaper 1 in which the urine indicators 50 are provided to two lateral sides of the feces indicator 40, it makes the sensor unit 80 easier to more accurately detect the change in the colors of the feces indicator 40 and the urine indicators 50.

In the sensor unit 80, the feces change-detection unit 812r and the urine change-detection units 822r are provided on the bottom face 80c, and the bottom face 80c is a surface to be attached to face the diaper 1. In some cases, this makes it difficult to determine the attachment position of the sensor unit 80 with respect to the feces indicator 40 and the urine indicator 50.

Therefore, the sensor unit 80 may include marks (M1, M2) that indicate positions for attaching the sensor unit 80 to the diaper 1, on the upper surface 80a, which is opposite to the side to be attached to the diaper 1 (bottom face 80c).

When the person replacing the diaper 1 attaches the sensor unit 80 to the diaper 1 according to the marks (M1, M2), it is possible to attach the sensor unit 80 at the correct position, making it possible for the replacing person to more accurately recognize whether defecation has occurred or urination has occurred.

Specifically, as shown in FIGS. 4 and 5 of one or more embodiments, the feces-indicator mark M1 and the urine-indicator marks M2 are provided as the marks (M1, M2).

The feces-indicator mark M1 and the urine-indicator marks M2 are provided on the upper surface 80a, which is opposite to the bottom face 80c.

The feces-indicator mark M1 is provided at a position corresponding to the feces change-detection unit 812r. In one or more embodiments, the feces-indicator mark M1 is provided at a position corresponding to the transparent portion 81, and has the same shape and size as the transparent portion 81 when viewed from the upper surface 80a side.

The feces-indicator mark M1 can have a predetermined shape, pattern, and color. For example, in one or more embodiments, the feces-indicator mark M1 is white and has a yellow mesh pattern.

The urine-indicator marks M2 are provided at a position corresponding to the urine change-detection units 822r. In one or more embodiments, the urine-indicator marks M2 are located at positions corresponding to the transparent portions 82, and have the same shape and size as the transparent portions 82 when viewed from the upper surface 80a.

The urine-indicator mark M2 can have a predetermined shape, pattern, and color. For example, in one or more embodiments, the urine-indicator mark M2 is white and has a yellow hatching pattern.

It should be noted that at least one of the shape, pattern, and color may be different between the feces-indicator mark M1 and the urine-indicator marks M2.

The person replacing the diaper 1 who has visually recognized the different marks (M1, M2) can easily distinguish the position corresponding to the feces indicator 40 and the positions corresponding to the urine indicators 50.

By providing the feces-indicator mark M1 and the urine-indicator mark M2, it is possible for the person replacing the diaper 1 to recognize the appropriate attachment position for the sensor unit 80. This makes it easier to attach the sensor unit 80 to the diaper 1, making it possible to detect the change in the colors of the feces indicator 40 and the urine indicators 50 at an accurate positions.

Further, the color of the feces-indicator mark M1 is set to white that is the identical color as the pre-change color of the feces indicator 40, and the color of the urine-indicator mark M2 is set to white that is the identical color as the pre-change color of the urine indicator 50.

This makes it easier to provide the marks (M1, M2) at the positions corresponding to the feces indicator 40 and the urine indicators 50.

It should be noted that in one or more embodiments, the pre-change color of the feces indicator 40 and the pre-change color of the urine indicator 50 are identical to each other. However, different colors may be used; for example the pre-change color of the feces indicator 40 is white and the pre-change color of the urine indicator 50 is yellow.

In such a case, the color of the feces-indicator mark M1 may be set to white that is the identical color to the pre-change color of the feces indicator 40, and the color of the urine-indicator mark M2 may be set to yellow that is the identical color to the pre-change color of the urine indicator 50.

By matching the marks (M1, M2) to the identical colors to the colors of the indicators 40 and 50, it is possible for the person replacing the diaper 1 to attach the sensor unit 80 at an appropriate position.

Further, the colors of the feces-indicator mark M1 and the urine-indicator marks M2 may be different from the post-change color of the feces indicator 40, and the colors of the feces-indicator mark M1 and the urine-indicator marks M2 may be different from the post-change color of the urine indicators 50.

In addition to the detection result by the sensor unit 80, there is a case where the person replacing the diaper 1 recognizes the change in the colors of the feces indicator 40 and the urine indicators 50 by directly visually recognizing the diaper 1.

At this time, making the colors of the feces-indicator mark M1 and the urine-indicator mark M2 different from the post-change color of the feces indicator 40 and the post-change color of the urine indicators 50 makes the person replacing the diaper 1 easier to recognize the change in the color of each of the indicators 40 and 50.

Further, as shown in FIG. 5, in the state where the sensor unit 80 is attached to the diaper 1, the vertical (first-direction) length of the sensor unit 80 may be shorter than the vertical length of the feces indicator 40, and the vertical (first-direction) length of the sensor unit 80 may be shorter than the vertical length of the urine indicators 50.

Therefore, even in the state where the sensor unit 80 is attached to the diaper 1, the person replacing the diaper 1 can directly visually recognize the feces indicator 40 and the urine indicator. Therefore, the replacing person can confirm whether the wearer has defecated or the wearer has urinated, with the detection result by the sensor unit 80.

Package 100

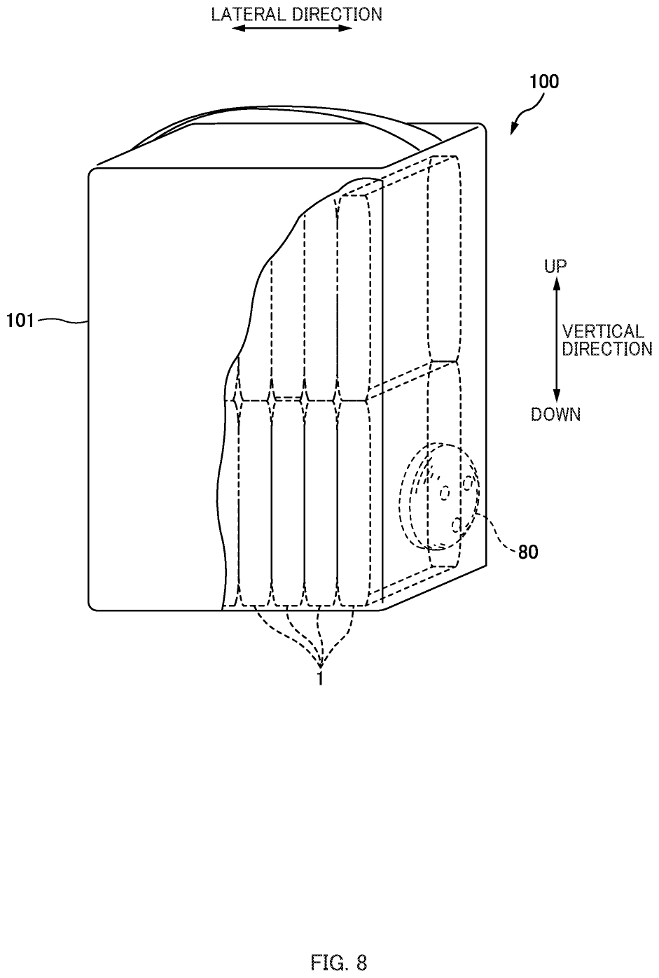

FIG. 8 is a perspective view of a package 100.

As shown in FIG. 8, in the package (absorbent-article packaged piece) 100, a plurality of diapers 1, 1, . . . , and sensor units 80 are accommodated in a packaging member 101 which is for packaging, the number of sensor units 80 being smaller than the number of the diapers 1.

In one or more embodiments, sixteen diapers 1 and one sensor unit 80 are accommodated in one package 100.

The number of the sensor units 80 is not limited to one for the sixteen diapers 1, and any number of the sensor units 80 from 1 to 15 can be accommodated.

The sensor unit 80 can be attached when putting on the diaper 1, and be detached and attached to a new diaper 1 when replacing the diaper 1.

That is, one sensor unit 80 can be attached to a plurality of diapers 1.

Therefore, in the package 100, a smaller number of sensor units 80 than the number of the diapers 1 can be accommodated, and it is possible to reduce the cost of the package 100 as a product.

### Other Embodiments

Although the above embodiments of the present invention have been described, but the above-described embodiments are intended to facilitate the understanding of the present invention and are not intended to limit the interpretation of the present invention.

In addition, the present invention can be modified or improved within the scope of the gist of the present invention, and additionally, it is needless to say that equivalents thereof are included in the present invention.

In the above-described embodiments, the sensor unit 80 is formed of a non-flexible plastic material, but the present invention is not limited thereto.

The sensor unit 80 may have flexibility.

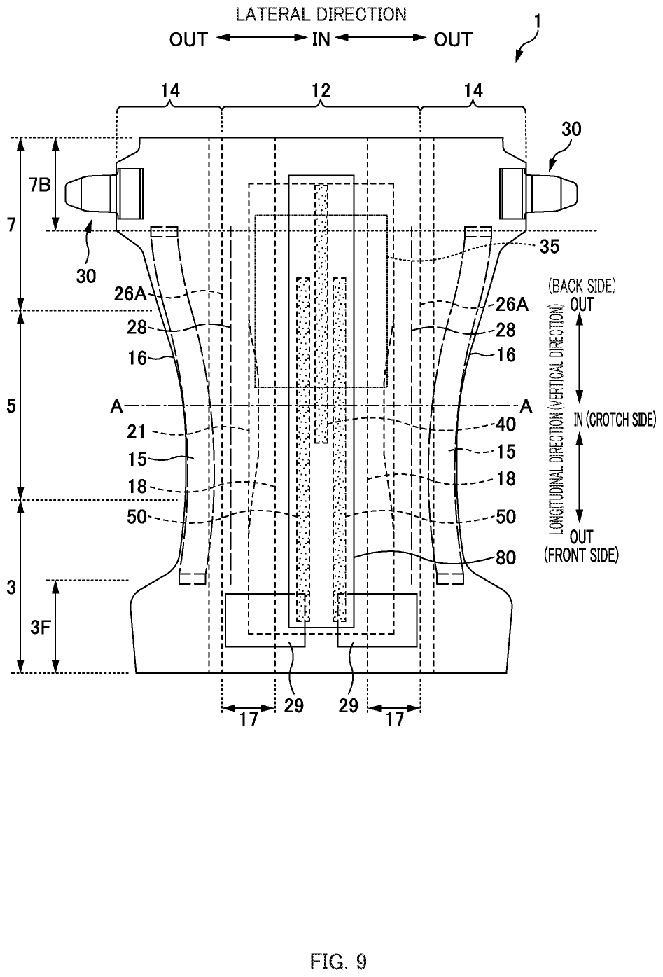

FIG. 9 is a diagram illustrating a modified example of the sensor unit 80.

FIG. 0.9 shows a flexible sheet-like sensor unit 80 and has a rectangular shape with a size that covers the entire region of the feces indicator 40 and the urine indicators 50.

For example, the sensor unit 80 can be formed by fixing the light sources 811e and 821e (both not shown), and the detection units 812r and 822r (both not shown) to a sheet member which is formed of a nonwoven fabric or nylon.

Since the sensor unit 80 has flexibility, the texture for the wearer is good, and there is a small risk of limiting the movement of the wearer.

Further, in the flexible sensor unit, the stiffness of the portion where the feces change-detection unit 812r and the urine change-detection unit 822r are not provided may be lower than the stiffness of the vertical central portion and the lateral central portion of the diaper 1.

Therefore, the texture to the wearer can be further enhanced.

In the above-described embodiments, the sensor unit 80 is attached to the wearer's front side), but the configuration is not limited thereto.

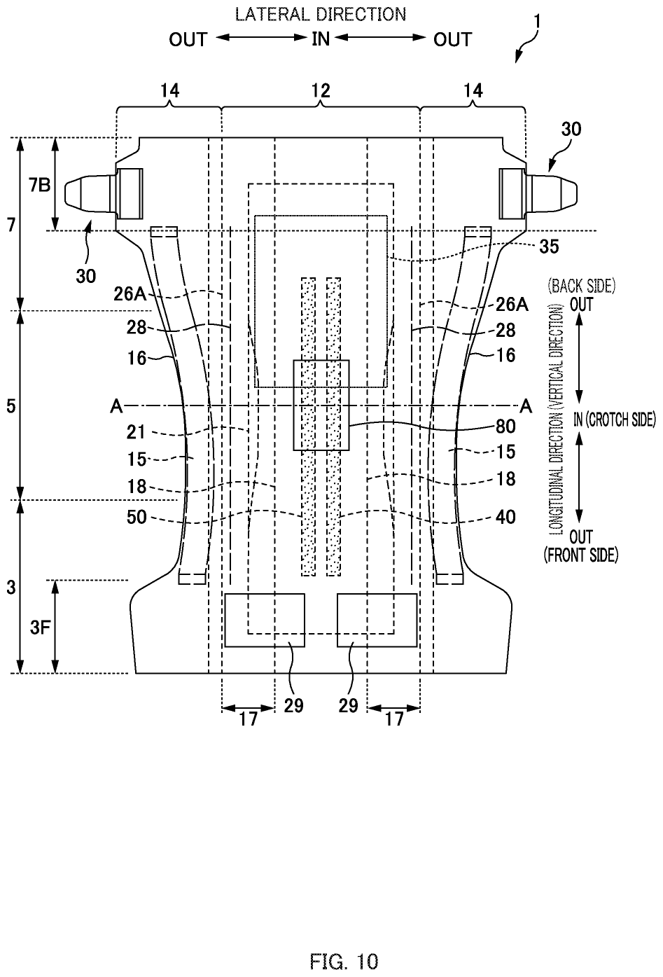

For example, as shown in FIG. 10, the absorbent article may be provided at the wearer's crotch portion, that is, over from the front side to the back side.

FIG. 10 is a diagram illustrating a modified example of the sensor unit 80.

In the case of being attached to a curved portion such as a crotch portion, the sensor unit 80 may have flexibility.

The attachment position of the sensor unit 80 can be determined with respect to the diaper 1, according to the wearer's body and posture.

Further, as shown in FIG. 10, the diaper 1 may include one feces indicator 40 extending along the vertical direction and one urine indicator 50 extending along the vertical direction.

The present invention is also applicable to absorbent articles (e.g., diapers) having different shapes and numbers of the feces indicator 40 and the urine indicator 50.

In the above-described embodiments, the sensor unit 80 outputs the detection result to the terminal in which wireless communication is set, and causes the detection result to be displayed on the screen of the terminal, but the present invention is not limited thereto.

The sensor unit 80 may output the output result to any terminal via a device that is different from the terminal (e.g., a server or a relay device).

In this case, the following is also acceptable: the output results are stored in the different device and the terminal obtains the output results that have been output from the different device.

Alternatively, the following is also acceptable: the accumulated information of the output results which has been accumulated as a result of multiple times of detection is stored in the terminal or a device different from the terminal, and the time and the amount of excretion (defecation, urination, or the like) are predicted from the stored accumulated information.

Further, in the above-described embodiments, the terminal displays on the screen of the terminal that the color of the feces indicator 40 is changed or the color of the urine indicators 50 are changed. But the present invention is not limited thereto.

For example, the user may be notified of defecation or urination by other output methods, namely sound through the speaker of the terminal (e.g., music or voices), smell, and light.

Note that it is sufficient that, regardless of whether the output unit is wired or wireless, the output unit outputs information to a device which is connected to the sensor unit 80.

Further, the information output by the output unit may be a signal detected by each of the detection devices 812 and 822 (each of the detection units 812r and 822r), or may be information obtained by performing signal processing on a signal detected by each of the detection devices 812 and 822 (each of the detection units 812r and 822r) in the sensor unit 80 (e.g., in the control unit 84).

In the above-described embodiments, the output unit wirelessly outputs the output result to any terminal, but the present invention is not limited thereto.

For example, the sensor unit 80 may be configured to include a speaker and to output the output result as a sound (alarm).

In this case, making different the sound used when outputting the detection result that the color of the feces indicator 40 is changed and the sound used when outputting the detection result that the color of the urine indicator 50 is changed makes it possible for the person replacing the diaper 1 to recognize whether defecation has occurred or excretion has occurred.

For example, the following configuration is acceptable: in the case where defecation occurs and the change in the color of the feces indicator 40 is detected by the feces change-detection unit 812r, the output unit outputs a voice saying “I got a poo” and in the case where urination occurs and the change in the color of the urine indicator 50 is detected by the urine change-detection unit 822r, the output unit outputs a voice saying “I got pee-pee”.

In such a case, the person replacing the diaper 1 can recognize without using any terminal or the like whether the wearer has excreted urine or the wearer has excreted feces.

Further, in the sensor unit 80, there may be installed sensors (detection units and detection devices) other than the feces change-detection device 812 (feces change-detection unit 812r) and the urine change-detection devices 822 (urine change-detection units 822r).

For example, the following devices may be included: a timepiece device that displays the time when the feces change-detection device 812 and the urine change-detection devices 822 respectively detect the change in the colors of the feces indicator 40 and the urine indicators 50; a measurement device that measures the position, the speed, the acceleration, the angle, the angular velocity, the angular acceleration of the sensor unit 80, the ambient temperature and humidity of the sensor unit 80, and the odor; and the like.

The information detected by these sensors (timepiece device and measurement device) may be output by the output unit.

In the above-described embodiments, the light sources 811e and 821e use three LED light beams (white light) of red (R), green (G), and blue (B), and the feces change-detection device 812 and the urine change-detection devices 822 are RGB color sensors. However, the present invention is not limited thereto.

For example, the light source 811e, the light sources 821e, the feces change-detection device 812, and the urine change-detection devices 822 may be monochromatic color sensors having sensitivities to the post-change color of the feces indicator 40 and the post-change color of the urine indicators 50.

In the above-described embodiments, as the attaching/detaching mechanisms 83, a member that is attachable to or detachable from the non-skin-side surface (exterior sheet 27) of the diaper 1 is used, but the present invention is not limited thereto.

For example, the attaching/detaching mechanism 83 and an attachable/detachable member may be separately attached to the non-skin-side surface of the diaper 1.

Further, the sensor unit 80 may be configured not to have the attaching/detaching mechanism 83 and to include a fixing member that non-detachably fixes the sensor unit 80 to the diaper 1.

In the above-described embodiments, the marks (M1, M2) indicating the positions at which the sensor unit 80 is attached to the diaper 1 are each circular, but the present invention is not limited thereto.

The marks (M1, M2) can have any shape.

For example, the feces-indicator mark M1 may have a shape formed along the shape of the feces indicator 40, and the urine-indicator mark M2 may have a shape formed along the shape of the urine indicator 50.

Further, in the above-described embodiments, the colors of the marks (M1, M2) are white, which is the identical color to the pre-change color of the feces indicator 40 and the pre-change color of the urine indicator 50. But the present invention is not limited thereto.

The colors of the feces-indicator mark M1 and the urine-indicator mark M2 may each be different from the pre-change color of the feces indicator 40 and the pre-change color of the urine indicator 50.

Further, the color of the feces-indicator mark M1 may be the identical color to the post-change color of the feces indicator 40, and the post-change color of the urine indicator 50 may be the identical color to the urine-indicator mark M2.

In such a case, the replacing person who directly visually recognizes the diaper 1 can recognize that defecation has occurred due to the color of the feces indicator 40 being identical to the color of the feces-indicator mark M1, and also can recognize that urination has occurred due to the color of the urine indicator 50 being identical to the color of the urine-indicator mark M2.

In the above-described embodiments, the package 100 is configured such that the plurality of diapers 1, 1, . . . , and a smaller number of sensor units 80 than the number of the diapers 1 are accommodated in the packaging member 101 for packaging, but the present invention is not limited thereto.

The package 100 in which the same number of sensor units 80 as the number of diapers 1 are accommodated may be used.

For example, the package 100 in which one diaper 1 and one sensor unit 80 are accommodated may be used.

Although the disclosure has been described with respect to only a limited number of embodiments, those skilled in the art, having benefit of this disclosure, will appreciate that various other embodiments may be devised without departing from the scope of the present disclosure.

Accordingly, the scope of the invention should be limited only by the attached claims.

### REFERENCE SIGNS LIST

1 Diaper (tape-type disposable diaper, absorbent article), 3 Front portion, 3F front waist portion, 5 Crotch portion, 7 Back portion, 7B back waist portion, 12 central band-shaped region, 14 side flap, 15 leg-gather elastic member, 16 leg gather, 17 leg side gather, 18 leg-side-gather elastic member, 21 Absorbent body, 22 Top sheet, 23 back sheet, 24 Absorbent core, 25 Core-wrapping sheet, 26 Skin-side sheet, 26A Joining portion, 27 Exterior Sheet, 28 leg elastic member, 29 Target tape, 30 Fastening tape, 35 Second sheet, 40 Feces indicator, 50 urine indicator, 80 Sensor unit, 81 Transparent portion, 811 Light emitting device, 811e Light source, 812 Feces change-detection device, 812r Feces change-detection unit, 82 Transparent portion, 821 Light emitting device, 821e Light source, 822 urine change-detection device, 822r urine change-detection unit, 83 Attaching/detaching mechanism, 84 Control unit, 85 Substrate, 100 Package (absorbent-article packaged piece), 101 Packaging member

What is claimed is:

1. A sensor unit for an absorbent article comprising:
a urine change-detection unit that detects a change in color of a urine indicator that visually changes a color on contact with urine; and
a feces change-detection unit that detects a change in color of a feces indicator that visually changes a color on contact with feces.

2. The sensor unit according to claim 1, further comprising:
a controller that wirelessly outputs detection results detected by the urine change-detection unit and the feces change-detection unit.

3. The sensor unit according to claim 1, wherein
the sensor unit is attached to the absorbent article such that, along a vertical direction of the absorbent article, a center of the urine change-detection unit is offset from a center of the feces change-detection unit.

4. The sensor unit according to claim 1, wherein
the sensor unit is attached to the absorbent article such that, along a lateral direction of the absorbent article, a center of the urine change-detection unit is offset from a center of the feces change-detection unit.

5. The sensor unit according to claim 1, wherein
the urine change-detection unit is disposed on each side of the feces change-detection unit in a lateral direction of the absorbent article.

6. The sensor unit according to claim 1, further comprising:
an attaching/detaching mechanism that attaches and detaches the sensor unit to and from the absorbent article, and
the urine change-detection unit and the feces change-detection unit each have a portion that does not overlap the attaching/detaching mechanism when viewed from a side from which the sensor unit is attached to the absorbent article.

7. The sensor unit according to claim 1, wherein
the sensor unit is flexible.

8. The sensor unit according to claim 1, wherein
the sensor unit has an attachment side to be attached to the absorbent article and a non-attachment side opposite the attachment side, and
the sensor unit has a mark, on the non-attachment side, that indicates an attachment position where the sensor unit is to be attached to the absorbent article.

9. The sensor unit according to claim 8, wherein
the mark includes:
a urine-indicator mark at a position corresponding to the urine change-detection unit, and
a feces-indicator mark at a position corresponding to the feces change-detection unit.

10. The sensor unit according to claim 9, wherein
the urine-indicator mark and the feces-indicator mark have graphics, and
at least one of shapes, patterns, and colors of the graphics are different between the urine-indicator mark and the feces-indicator mark.

11. The sensor unit according to claim 9, wherein
a color of the urine-indicator mark is identical to a color of the urine indicator before the urine indicator changes the color on contact with the urine, and
a color of the feces-indicator mark is identical to a color of the feces indicator before the feces indicator changes the color on contact with the feces.

12. The sensor unit according to claim 8, wherein
a color of the mark is different from a color of the urine indicator after the urine indicator changes the color on contact with the urine, and
the color of the mark is different from a color of the feces indicator after the feces indicator changes the color on contact with the feces.

13. An absorbent article kit comprising:
an absorbent article; and
a sensor unit, wherein
the absorbent article comprises:
a urine indicator that:
visually changes a color on contact with urine, and extends along a vertical direction of the absorbent article; and
a feces indicator that:
visually changes a color on contact with feces, and extends along the vertical direction, and
the sensor unit comprises:
a urine change-detection unit that detects a change in color of the urine indicator; and
a feces change-detection unit that detects a change in color of the feces indicator.

14. The absorbent article kit according to claim 13, wherein
the sensor unit has an attachment side to be attached to the absorbent article,
in a state where the sensor unit is attached to the absorbent article while the attachment side facing the urine indicator and the feces indicator,
a vertical length of the sensor unit is shorter than a vertical length of the urine indicator, and
the vertical length of the sensor unit is shorter than a vertical length of the feces indicator.

15. The absorbent article kit according to claim 13, wherein
the sensor unit is flexible, and
a stiffness of a portion of the sensor unit in which the urine change-detection unit and the feces change-detection unit are not disposed is lower than a stiffness of a vertical central portion and a lateral central portion of the absorbent article.

16. An absorbent-article package comprising:
a packaging medium;
a plurality of absorbent articles; and
a sensor unit, wherein
the absorbent articles and the sensor unit are accommodated in the packaging medium,
a number of the sensor unit is smaller than a number of the absorbent articles,
each of the absorbent articles comprises:
a urine indicator that:
visually changes a color on contact with urine, and extends along a vertical direction of the absorbent article; and
a feces indicator that:
visually changes a color on contact with feces, and extends along the vertical direction, and
the sensor unit comprises:
a urine change-detection unit that detects a change in color of the urine indicator; and
a feces change-detection unit that detects a change in color of the feces indicator.
